(19) United States
(12) Patent Application Publication
(10) Pub. No.: US 2015/0045322 A1
(43) Pub. Date: Feb. 12, 2015

(54) COMBINATION TREATMENT COMPRISING SULPHATED GLYCOSAMINOGLYCANS FOR INDUCING LABOR

(71) Applicant: DILAFOR AB, Solna (SE)

(72) Inventors: Gunvor EKMAN-ORDEBERG, Danderyd (SE); Anders MALMSTRÖM, Lund (SE)

(21) Appl. No.: 14/387,936

(22) PCT Filed: Mar. 25, 2013
(86) PCT No.: PCT/SE2013/050332

§ 371 Date: Sep. 25, 2014

Related U.S. Application Data
(60) Provisional application No. 61/615,398, filed on Mar. 26, 2012.

(51) Int. Cl. A61K 31/737; A61K 31/5575; A61K 31/727

(52) U.S. Cl. CPC A61K 31/737; A61K 31/727; A61K 31/5575
USPC 514/56

(57) ABSTRACT

The present invention refers to the use of certain sulfated glycosaminoglycans for inducing labor. The sulfated glycosaminoglycans have a reduced anticoagulant activity and are used in a combination therapy together with treatment capable of promoting cervical ripening or capable of promoting myometrial contractions of the uterus.

### FIELD OF INVENTION

The present invention refers to the use of certain sulfated glycosaminoglycans for inducing women into labor.

### BACKGROUND

It is a common clinical situation in obstetrics that labor needs to be induced due to an extended pregnancy, for example beyond the 41-42 weeks gestation time, or due to numerous medical complications, exemplified by pre-eclampsia, diabetes, essential hypertonia and Intra Uterine Growth Retardation (IUGR).

At labor induction the cervix not seldom is unripe as a result of insufficient remodeling of the cervical extra cellular matrix (ECM). An insufficient remodeling of the uterine ECM with low concentration of heparan sulfate is associated with dystocia. A normal cervical ripening and dilation of the cervix opening from 1 to 10 cm during established labor implies a total reconstruction of the cervical ECM with an inflammatory reaction resulting in a decrease of the concentration of collagen and proteoglycans. Disturbances in the cervical ripening can, if the process starts too early, result in a pre-term delivery. On the other hand, insufficient cervical ripening may result in post-term delivery with high frequencies of protracted labor and, as a consequence, instrumental deliveries. Thus, cervical ripening and myometrial contractions are two processes, which must be coordinated to accomplish a normal delivery.

Labor can be induced in a number of ways. Non-limiting examples of methods to induce labor are physical stimulation processes; administration of oxytocin, prostaglandin E or derivatives thereof, such as misoprostol and dinoproston; rupturing the amniotic sac; expanding the cervix, administrating an intracervical balloon and use of intra cervical Foley catheter (providing an endogenous release of prostaglandin from decidua and cervix). Also combinations of these labor inducing processes can be used. Even if it is common practice to administer these agents or processes to induce labor, it is a fact that women subjected to labor induction suffer from frequent incidences of labor dystocia, including labor arrest, prolonged latent phase of labor and slow progress of labor (protracted labor). It is also estimated that 15-20% of the interventions to induce labor in women with unfavorable cervices fail following local application of prostaglandin E2. Despite these facts, there have been few efforts to develop new drugs aiming at improving labor induction and the following events until delivery. The failure in establishing reliable and safe treatments has resulted in an increasing number of Caesarean sections and operative deliveries.

In Acta Obstetricia et Gynecologica. 2010; 89: 147-150) it is reported that dalteparin, a Low Molecular Weight Heparin (LMWH) has been found to improve labor progress and thereby reduce the labor time and it is suggested that dalteparin increases the oxytocin induced uterine smooth muscle contractions and also stimulate the release of cytokines in cervical cells cultivated from biopsies taken from cervix at partus. Even if dalteparin generally appears to cause positive effects on the labor process, it would not be clinically feasible to use due to the risks for bleeding from its anticoagulant effect.

WO 03055499 teaches that sulfated glycosaminoglycans, such as heparin, having an anticoagulant activity of 100 BP units/mg or less, are effective for prophylactic priming or curative treatment of the cervix and the myometrium for establishing effective labor in women in general. In this document, it is suggested that sulfated glycosaminoglycans can be used in combination with oxytocin for the priming of the myometrium in cases of low endogenous oxytocin levels. It is however, not suggested that the sulfated glycosaminoglycans would be useful in directly intervening therapies when complications arise that require a direct therapeutic efficacy.

There is a need for an agent that can be used as a support to existing therapies to induce labor in women elected for a direct intervention therapy to enter into labor. It thereby would be desirable to provide a therapy with a rapid onset that both can contribute to the process of establishing cervical ripening of an unripe cervix and to promote myometrial contractions of the uterus, so as to avoid or eliminate any of the complications associated with labor dystocia.

### DESCRIPTION OF THE INVENTION

Before the present invention is described, it is to be understood that the terminology employed herein is used for the purpose of describing particular embodiments only and is not intended to be limiting, since the scope of the present invention will be limited only by the appended claims and equivalents thereof.

It must be noted that, as used in this specification and the appended claims, the singular forms “a,” “an,” and “the” include plural referents unless the context clearly dictates otherwise.

Also, the term “about” is used to indicate a deviation of +/−2% of the given value, preferably +/−5%, and most preferably +/−10% of the numeric values, where applicable.

In the context of the present invention “labor induction” is generally defined as an intervention that directly or indirectly onsets labor from myometrial contractions of the uterus (uterine contractions) to accomplish a progress resulting in delivery and childbirth. The reasons for inducing labor include, but are not limited to, an extended pregnancy for example beyond the 41-42 weeks gestation time or medical complications, exemplified by pre-eclampsia, diabetes, essential hypertonia and Intra Uterine Growth Retardation (IUGR). Apart from many well practiced processes, labor induction is conventionally triggered by administration of prostaglandins, such as dinoproston and by administration of oxytocin.

In the context of the present invention the term “inducing labor” relates to a therapy where a direct response effect is requested from the administration. In the context of labor it is requested that the administration directly leads to at least one of initiation of cervical ripening or promotion or stimulation of uterine contractions. In other terms, the present invention is not directed to a prophylactic therapy, wherein women may receive a therapy to prevent from or counteract protracted labor, before being elected for labor induction.

The term “elected for labor induction” has the meaning that the pregnant woman has been elected for a clinical reason, as outlined with “labor induction”, or a humanitarian reason to enter into labor and that the labor shall be induced with a directly intervening administration therapy that directly after the administration initiates a process that directly or indirectly leads to the onset of labor. In the context of the present invention, the process leading to the onset of labor can include at least one of initiation or promotion of cervical ripening or promotion or stimulation of myometrial contractions of the uterus.

The terms “dystocia” or “labor dystocia”, as used in the context of describing the present invention, are general terms covering several conditions including labor arrest, prolonged latent phase of labor and slow progress of labor (protracted labor). Dystocia is particularly common after labor induction and more frequent among nulliparous than multiparous women.

The term “combination treatment” or “treatment in combination” is herein defined as a treatment with a chemically modified heparin or heparan sulfate described and claimed herein and another treatment that is effective to accomplish labor induction. The other treatment is a different treatment that is effective in promoting cervical ripening or myometrial contractions of the uterus. The other treatment can include administration of an agent capable promoting cervical ripening or myometrial contractions of the uterus, or it can include invasive and non-invasive treatments that for example can trigger and endogenous release of prostaglandins contributing to labor induction. Skilled obstetricians are aware of a number of such treatments. A combination treatment may include that a treatment with chemically modified heparin or heparan sulfate described and claimed herein is performed adjunctively, simultaneously or sequentially with the other treatment. It may also have the meaning of a chemically modified heparin or heparan sulfate described in the present invention administered as an add-on therapy to another treatment useful to induce labor. In the aspect when the combination treatment is an add-on therapy, the administration of a chemically modified heparin or heparan sulfate is added to another treatment for inducing labor at any time after initiating the other therapy.

Sulfated glycosaminoglycans with low anticoagulant effect, such as an anti-factor Xa activity below 200 IU/mg are disclosed herein for use in inducing labor.

The glycosaminoglycans are sulfated glycosaminoglycans selected from the group consisting of heparan sulfate, depolymerised heparan sulfate, dermatan sulfate, depolymerised dermatan sulfate, heparin, depolymerized heparin (low molecular weight heparin), chondroitin sulfates and depolymerised chondroitin sulfates.

The sulfated glycosaminoglycans are heparan sulfate, heparin, dermatan sulfate and chondroitin sulfate, which are composed of alternating hexosamine and uronic acid residue. The presence of D-glucuronic acid (GlcA) and its C-5 epimer L-iduronic acid (IdoA) and the specific sulfation of hexosamines and uronosyl residue endow the polymer an extreme structural variation. The structure is built on repeating disaccharides containing from none or very few to nearly 100% iduronic acid-containing disaccharides. The organization of GlcA-and IdoA-N-hexosamine containing disaccharides can vary from long blocks to an alternating disaccharide pattern. The variation of sulfation and the degree of iduronic acid sulfate generates a wide variety of biological activity. There are different well-defined polysaccharides of dermatan sulfate, chondroitin sulfate, heparan sulfate and depolymerised heparin.

Chondroitin sulfate is a sulfate linear polysaccharide consisting of alternating glucuronic acid and N-acetyl-galactosamine residue, the latter being sulfate in either 4 or 6 position. They can be prepared from bovine trachea or nasal cartilage. Chondroitin sulfate is of importance for the organization of extracellular matrix, generating a interstitial swelling pressure and participating in recruitment of neutrophils.

Dermatan sulfate is a sulfate linear polysaccharide consisting of alternating uronic acid and N-acetyl-galactosamine residue. The uronic acids are either D-GlcA or L-IdoA and the disaccharide can be sulfate in 4 and 6 and 2 on galactosamine and IdoA, respectively. Dermatan sulfate can be prepared from porcine skin or intestinal mucosa and bovine lung, possesses biological activities such as organization of extracellular matrix, interactions with cytokines, anticoagulant activities and recruitment of neutrophils.

Heparan sulfate, having glucosamine and uronic acid as repeating disaccharides and consisting of N-acetylated and N-sulfated disaccharides that are arranged mainly in a segregated manner, has ubiquitous distribution on cell surfaces and in the extracellular matrix. It is generally less sulfate and has a lower iduronate content than heparin and has a more varied structure. Interactions between heparan sulfate and proteins are implicated in a variety of physiological processes, such as cell adhesion, cell proliferation, enzyme regulation, cytokine action, virus entry and anticoagulant properties. Heparan sulfates possess anticoagulant activity depending on the presence of a specific anticoagulant pentasaccharide, however considerably less than heparin. Heparan sulfate is a linear polysaccharide which can be prepared from porcine intestinal mucosa or from bovine lung, from heparin side fractions using cetylpyridinium chloride fractions and sequential salt extraction as described by Fransson et al., Structural studies on heparan sulphates, Eur. J. Biochem. 106, 59-69 (1980).

Heparin is a naturally occurring glycosaminoglycan that is a potent anticoagulant and has been used clinically for more than 60 years as the drug of preference for prophylaxis and treatment of thromboembolic disorders. The major potential adverse effects of heparin treatment are bleeding complications caused by its anticoagulant properties. Heparin is highly polydisperse and composed of a heterogeneous population of polysaccharides with molecular weights ranging from 5 to 40 kDa, with the average being approximately 15 to 18 kDa. Low molecular weight heparin or depolymerised heparin is linear oligosaccharides mainly consisting of alternating N-sulfated glucosamine and IdoA residue and often containing the anticoagulant pentasaccharide. They can be prepared from heparin by specific chemical cleavage. Their main clinical function is to inhibit factor Xa, resulting in an antithrombotic effect. It is proposed to have antimetastatic properties. Fragmin® (Pfizer, USA) is an example of a low molecular heparin obtained by controlled depolymerization of heparin and having an antithrombotic effect owing to inhibition of factor Xa. Heparin fragments having selective anticoagulant activity, as well as methods for the preparation thereof, are described in U.S. Pat. No. 4,303,651. According to the European pharmacopoeia (PharmEur) a heparin in order to be called a low molecular weight heparin (low molecular mass heparin) should have an antifactor Xa activity not less than 70 IU(International Unit)/mg and an M_{w} of less than 8 000 Da. The anticoagulant activity of heparin, Low Molecular Weight Heparins and other heparin derivatives is often measured as their ability to potentiate the inhibition of coagulation factor Xa and factor IIa by antithrombin. Methods for measuring anti-factor Xa- and anti-factor IIa activity are well known to the skilled person and are also described in pharmacopoeias such as the European pharmacopoeia (Pharm Eur) and the United States Pharmacopoeia (USP). The anticoagulant activity can be abrogated by for example selective periodate oxidation (see e.g. Fransson L A, and Lewis W, Relationship between anticoagulant activity of heparin and susceptibility, to periodate oxidation, FEBS Lett. 1979, 97:119-23; Lindahl et al., Proc Natl Acad Sci USA, 1980; 77(11):6551-6555) but also by other means known to the skilled person.

In one aspect, the invention relates to a chemically modified heparin or heparan sulfate with an antifactor II activity of less than 10 IU/mg, an antifactor Xa activity of less than 10 IU/mg comprising:

(i) polysaccharide chains essentially free of chemically intact saccharide sequences mediating the anticoagulant effect; and

(ii) polysaccharide chains corresponding to molecular weights between 1.2 and 12 kDa with a predominantly occurring disaccharide according to (Formula I),

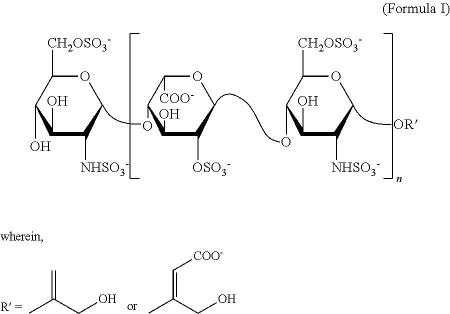

n is an integer from 2 to 20
   for use in combination with a treatment capable of promoting cerivcal ripening or promoting myometrial contractions for the induction of women into labor.

In this context, a chemically modified heparin or heparin sulfate, comprising polysaccharide chains essentially free of chemically intact saccharide sequences mediating the anticoagulant effect means that the polysaccharide chains have been treated chemically to modify essentially all the pentasaccharides specifically mediating an anticoagulant effect by antithrobmin (AT).

In one aspect the treatment comprises at least one of administration of an agent effective in promoting cervical ripening or an agent of effective in promoting myometrial contractions of the uterus.

In one aspect, the chemically modified heparin or heparan sulfate is used an add-on therapy to a treatment capable of promoting cervical ripening or promoting myometrial contractions of the uterus.

In one aspect, the treatment comprises at least one of rupturing the amniotic sac (amniotomy); expanding the cervix, administrating an intracervical balloon and using an intracervical Foley catheter (providing an endogenous release of prostaglandin from decidua and cervix). According to this aspect, the treatment can include other methods or means to trigger the release of endogenous prostaglandins in order to promote induction of labor.

In one aspect, the use of the chemically modified heparin or heparin sulfate is directed to women who are elected to be induced into labor belong to a patient group associated with risks for clinical complications for the woman or the fetus/neonate, or the women can be elected for humanitarian reasons. Patient groups include women in an extended pregnancy beyond 41-42 weeks gestation time, women suffering from medical complications, such as pre-eclampsia, diabetes, essential hypertonia and Intra Uterine Growth Retardation (IUGR).

In one aspect, the invention relates to the defined chemically modified heparin or heparan sulfate for use in a combination with a treatment for promoting cervical ripening in women with an unripe cervix. In one aspect, promoting cervical ripening comprises administration of a prostaglandin, Prostaglandins and prostaglandin derivatives are commonly used or suggested as agents to promote cervical ripening

In one aspect and may be administered intravaginally, endocervically or extra-amniotically. In one aspect, the prostaglandin is selected from the group consisting of dinoprostone (PGE2) and misoprostol (PGE1). Also other prostaglandins or derivatives thereof can be useful, such as PGF2α, or agents like anti-progestines.

The state of cervix can be established by routine methods among obstetricians, such as Bishop's Score (cervix score). It is well established that women with a Bishop's Score of 5 or less have an unripe cervix. Conventional therapies to establish cervical ripeness with PGE2 include administration every 12 hours at the most four times. One commonly employed way estimating ripeness is to estimate cervical dilation. A dilation of 4 cm or more can be considered to manifest a ripe cervix.

In one aspect, the treatment comprises administration of agent capable of promoting or stimulating myometrial contractions. In one aspect, the agent is administered to women for inducing labor in women who have a ripe cervix, but who are absent of myometrial contractions of the uterus. The women according to this aspect can have undergone a combination treatment with a chemically modified heparin or heparin sulfate as earlier described or undergone treatment for promoting cervical ripening, such as receiving as a prostaglandin, or spontaneously obtained a ripened cervix as determined by according to routine methods performed by an obstetrician.

In one aspect, the agent capable of promoting or stimulating uterine contractions is oxytocin.

In one aspect, the invention is directed to the uses of a chemically modified heparin or heparan sulfate with an average molecular weight (Mw) from about 4.6 and 6.9 kDa.

In one aspect, the predominantly occurring polysaccharide chains of the chemically modified heparin or heparan sulfate have between 6 and 12 disaccharide units with molecular weights from 3.6 to 7.2 kDa,

In one aspect, the invention the chemically modified heparin or heparan sulfate has been treated with periodate in order to eradicate anticoagulant effects by eliminating antithrombin III binding affinities. One non-limiting way of obtaining such a chemically modified heparin or heparan sulfate is subjection to periodate oxidation followed by alkaline β-elimination of the product. This process leads elimination of the anticoagulant activity. The process disclosed in U.S. Pat. No. 4,990,502 (Lormeau et al) demonstrates one way of treating native heparin to selectively cleave residues of the pentasaccharide residues responsible for the anticoagulant effect and a following depolymerization that results in a low anticoagulant, low molecular weight heparin with a an average molecular weight 5.8 to 7.0 kDa.

In one aspect, at least 70% of the polysaccharide chains of the chemically modified heparin or heparan sulfate have a molecular weight above 3 kDa. The distribution of polysaccharides and their corresponding molecular mass expressed as cumulative % of weight can be according to the table:

| Molecular mass, | |
|---|---|
| kDa | Cumulative weight, % |
| >10 | 4-15 |
| >8 | 10-25 |
| >6 | 22-45 |
| >3 | >70 |

Furthermore, the polysaccharide comprises saccharide chains can have the reduced end residue as shown in Formula I and are essentially free of intact non-sulfated iduronic and/or glucuronic acids.

In one aspect, this chemically modified heparin or heparan sulfate comprises modified glucosamines present as signals in the interval of 5.0 to 6.5 ppm in a ¹H-NMR spectrum with an intensity (% ratio) of less than 4% in relation to the signal at 5.42 ppm from native heparin. These glucosamine signals may be present at 6.15 ppm and 5.95 ppm. In one aspect, less than 1% of the total content of glucosamines is modified.

In this context, “modified glucosamines” have the meaning of glucosamines with a residue structure not expected to be found in a ¹H-NMR spectrum from heparin products or low molecular weight heparin products (depolymerized heparins). The appearance of modified glucosamines may be attributed to the chemical modification process for oxidizing non-sulfated iduronic and/or glucuronic acid in order to substantially eliminate the anticoagulant effect. It is desirable to minimize the presence of modified glucosamines as they may represent unpredictable characteristics of the chemically modified heparin or heparan sulfate product, such as depolymerization upon storage.

In one aspect, the chemically modified heparin or heparan sulfate comprises modified glucosamines in the non-reducing ends with unsaturated bonds. Such modified glucosamines are present as signals at 5.95 ppm and 6.15 ppm in an ¹H-NMR spectrum.

In a further aspect, the present invention relates to a method of inducing labor in women, comprising administering an effective amount of any of the earlier defined chemically modified heparin or heparan sulfate in combination with another treatment of inducing labor. In this aspect, other treatments of inducing labor conform with what has been defined or discussed in earlier sections of the specification.

In one aspect of the method the women have an unripe cervix and comprises administration of an agent or performing a therapy capable of promoting cervical ripening, such as a prostaglandin. In an example of this aspect of the invention, the chemically modified heparin or heparan sulfate is administered intravenously or subcutaneously every 2 to 6 hours combined with a treatment with PGE2 for up to 12 to 48 hours, or every 4 hours combined with a treatment with PGE2 for up to up to 36 to 48 hours.

In one aspect of the method, the women elected for labor induction in women have established cervical ripening but suffer from insufficient, or are absent of, uterine contractions. In this aspect, the method comprises administration of an agent capable of promoting myometrial contractions of the uterus, such as oxytocin. In a non-limiting example of this aspect of the invention, the chemically modified heparin or heparan sulfate is administered at least once every 24 hours and adjunctively with a treatment with oxytocin for up to about 36 hours. In another aspect the chemically modified heparin or heparan sulfate is administered 1-24 times/24 h. In yet another aspect the chemically modified heparin or heparan sulfate is administered 6 times/24 h. In an example of this aspect of the invention a chemically modified heparin or heparan sulfate is administered intravenously or subcutaneously every 4 hour combined with oxytocin. In one aspect the chemically modified heparin or heparan sulfate is administered by continuous infusion. Under current clinical practice oxytocin is administered intravenously.

In one aspect of the method, the women receive up to 1.5 g of the chemically modified heparin or heparan sulfate per 24 h. In another aspect, the women receive up to 1.2 g of the chemically modified heparin or heparan sulfate per 24 h and as a non-limiting example the 1.2 g/24 h is administered 6 times in doses of 200 mg.

In one aspect of the method, the women have established cervical ripening from administration of the chemically modified and/or an agent capable of promoting cervical ripening, such as a prostaglandin but are not entering into labor due to absence of myometrial contractions of the uterus. In this aspect, the method of inducing labor comprises administration of the chemically modified heparin or heparan sulfate combined with an agent capable of promoting or stimulating uterine contractions, such as oxytocin.

The methods can comprise administration of the chemically modified heparin or heparan sulfate having the features as defined in any part of this specification.

In another aspect, the invention relates to the use of a chemically modified heparin or heparan sulfate, as defined in any section of this specification, for the manufacture of a medicament for treatment in a combination therapy to induce women into labor. The treatments conform with definitions in earlier sections of this specification.

The chemically modified heparin or heparan sulfate to be used with the invention can be administered systemically as pharmaceutical compositions by parenteral administration, such as by subcutaneous or intravenous injection. For parenteral administration the active compounds can be incorporated into a solution or suspension, which also contain one or more adjuvants such as sterile diluents such as water for injection, saline, fixed oils, polyethylene glycol, glycerol, propylene glycol or other synthetic solvents, antibacterial agents, antioxidants, chelating agents, buffers and agents for adjusting the osmolarity. The parenteral preparation can be delivered in ampoules, vials, disposable syringes or as infusion arrangements, also for self administration.

The chemically modified heparin or heparan sulfate to be used with the present invention can be administered subcutaneously and thereby with suitable self-administration tools, such as injectors.

Further, the chemically modified heparin or heparan sulfate to be used with the invention can be administered for topically by penetration of mucus membranes such as, but not limited to, vaginal, rectal, intrauterine, and nasal administration.

In one aspect, the chemically modified heparin or heparan sulfate to be used with the invention can be formulated together with an effective amount of an agent capable promoting cervical ripening or promoting myometrial contractions of the uterus and thereby be administered in together (co-administered) in one composition by previously suggested administration routes.

In one aspect, a composition of the chemically modified heparin or heparan sulfate to be used with the invention is included in a kit with at least one of a composition of an agent capable promoting cervical ripening and a composition promoting myometrial contractions of the uterus. The compositions can be provided in single or multidose forms adapted to different clinical situations. The dose forms can be adapted to administration tools which also may be a part of the kit. For this purpose, the kit can further comprise clinical instructions how and when to administer the included compositions.

By inducing labor in accordance with the invention, a shortened delivery time and the number of labor complications, e.g. Caesarian sections can be significantly reduced. Protracted labor is also associated with other maternal complications e.g. post partum haemorrhage, instrumental deliveries and endometritis as well as an increased risk of fetal asphyxia and infection. Oxytocin's lack of effect on the uterine contractility results in frequent cesarean sections, including the ones performed on an emergency basis.

Oxytocin is often administered to women in labor to establish or re-establish effective labor. Frequently, the oxytocin effect is impaired, probably due to a lack of adequate tissue levels of heparan sulfates leading to an overdosage of oxytocin that may result in severe side effects such as hypercontractility. The uses and methods method according to the present invention comprising administration of a chemically modified heparin or heparan sulfate can reverse the impaired oxytocin effect and thereby provide an oxytocin sparing effect and prevent the myometrial hypercontractility and as a consequence the risk of fetal complications.

According to current practice the concentration of the agent promoting myometrial contractions is titrated in order to reach the desired effect and to not administer more than necessary of said agent to the woman. The titration usually starts with a low dose which is increased until the desired effect (i.e. myometrial contractions of the uterus) has been established. In one aspect, a composition of the chemically modified heparin or heparan sulfate is included in a kit together with a multidose form comprising a composition comprising an agent capable of promoting myometrial contractions of the uterus adapted to admit administration in several doses. In one example, the kit comprises a multidose form of oxytocin and the chemically modified heparin or heparan sulfate is administered in combination with an initial low or standardized dose of oxytocin. Should the patient remain in labor arrest, oxytocin may be administered one or several times with controlled doses from the multidose form until progress of labor is re-established.

The chemically modified heparin or heparan sulfate may be effective by replenishing myometrial tissue levels such that it supports oxytocin to establish contractile effect on the myometrium with the effect that reduced amount of oxytocin may be administered, thereby reducing its negative side effects. Interestingly, it may be that the chemically modified heparin or heparan sulfate in the absence of oxytocin does not trigger any or only few myometrial contractions.

In accordance with the invention, the chemically modified heparin or heparan sulfate can exert its effect both on the cervix and on the uterus. With regard to cervical ripening the chemically modified heparin or heparan sulfate according to the invention can exert an effect together with prostaglandin E2 or other prostaglandins or prostaglandin derivatives useful to promote cervical ripening.

Encompassed by the present invention is any combination of the disclosed embodiments.

The invention will be further disclosed in the following non-limiting examples

### DETAILED AND EXEMPLIFYING DESCRIPTION

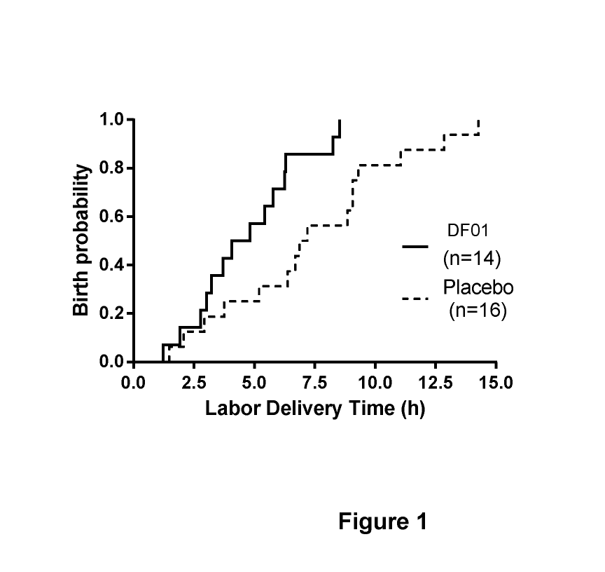

FIG. 1 shows delivery times in induced women who have been treated with a chemically modified heparin or heparan sulfate according to the invention and induced women who received placebo.

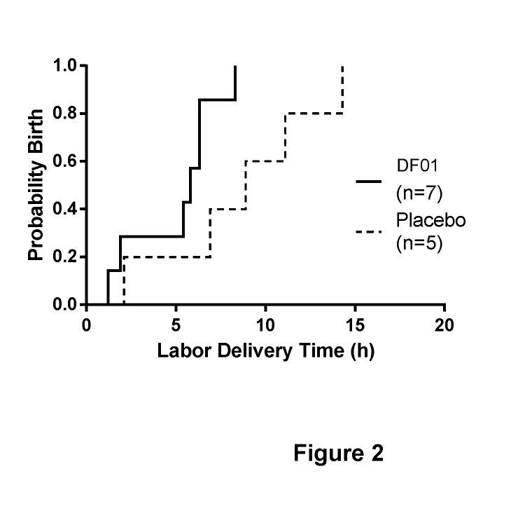

FIG. 2 shows delivery times in women who have been induced with prostaglandin E2 and have been treated with a chemically modified heparin or heparan sulfate according to the invention in comparison with women who have been induced with prostaglandin E2, but received placebo

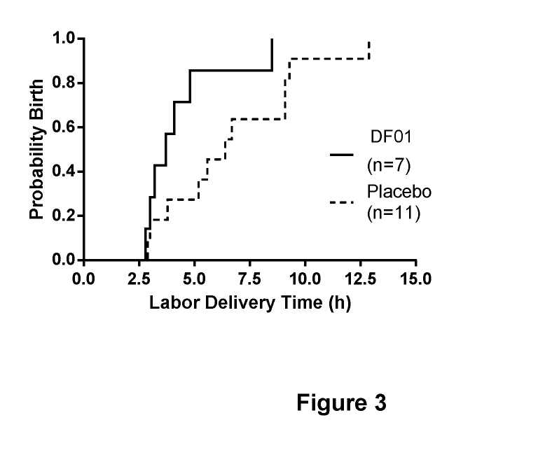

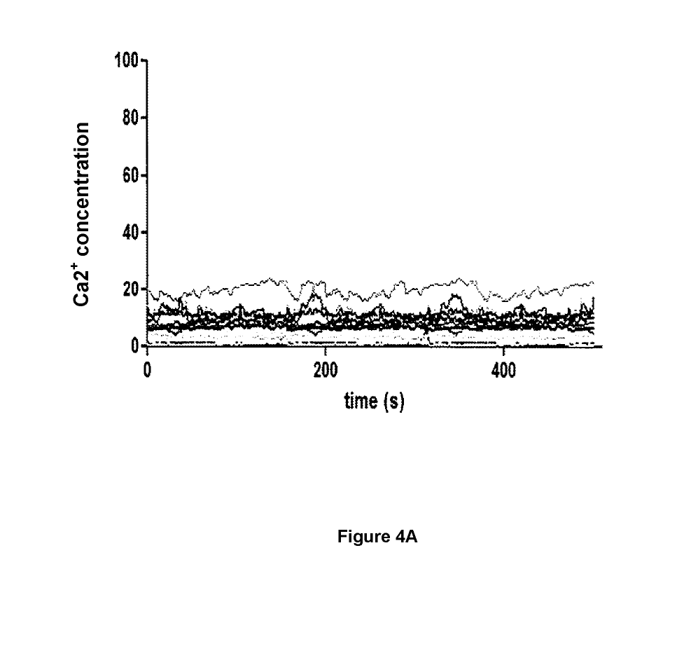

FIG. 3 shows delivery times in women who have been induced into labor with oxytocin and have been treated with a chemically modified heparin or heparan sulfate according to the invention in comparison to women who have been induced into labor with oxytocin, but received placebo.

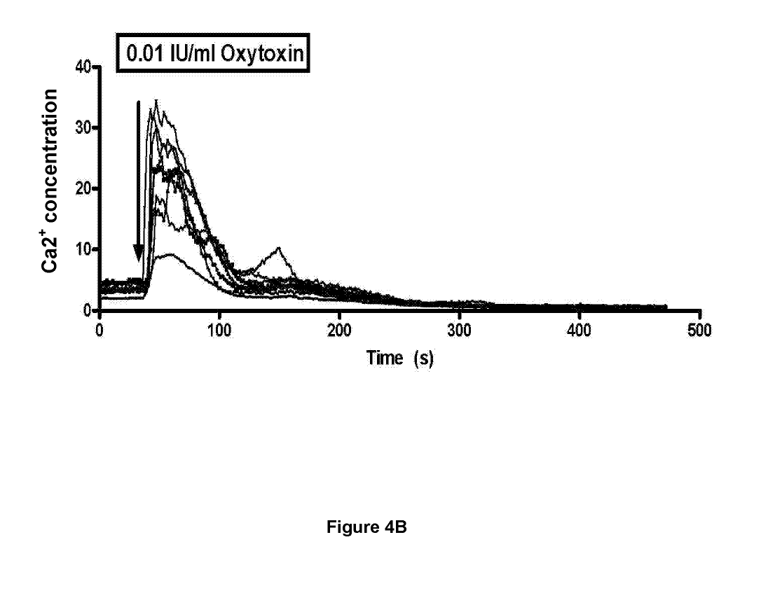

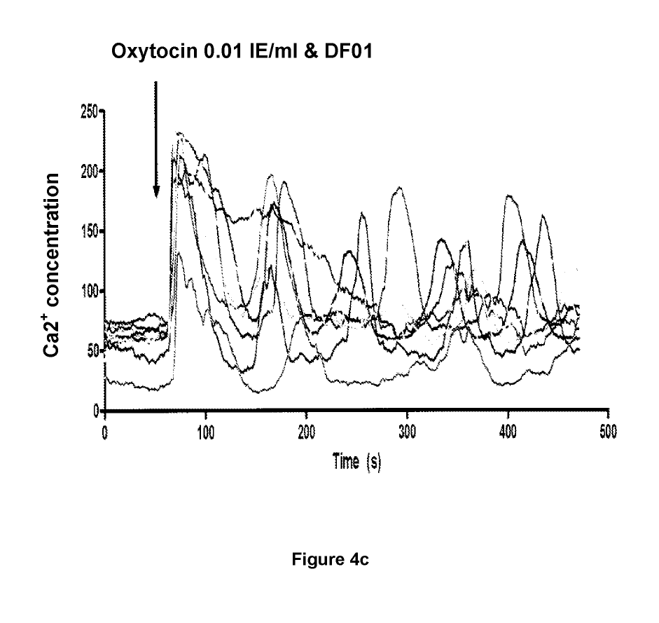

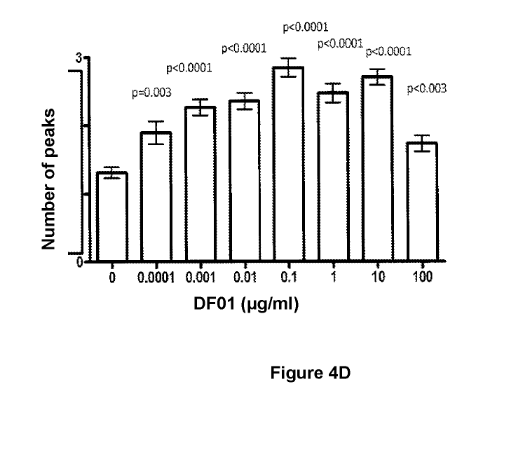

FIGS. 4A-4D show calcium ion influx in uterine muscle cells when treated with combinations of oxytocin and a chemically modified heparin or heparan sulfate according to the invention.

### EXAMPLES

Detailed Description of the Manufacturing Process of a Chemically Modified Heparin or Heparan Sulfate According to the Invention.

The following examples 1 to 9 serve as examples how to produce chemically modified heparin or heparan sulfates for use according to the present invention.

The substance is prepared from Heparin Sodium. The preparation involves selective oxidation of non-sulfated uronic acid residues in heparin by periodate, including the glucuronic acid moiety in the pentasaccharide sequence that binds AT. Disruption of the structure of this residue annihilates the high-affinity interaction with AT and, consequently, the anticoagulant effect (measured as a-FXa or a-FIIa) is essentially depleted. Subsequent alkaline treatment, beta-elimination reaction results in cleavage of the polymer at the sites of non-sulfated uronic acids that have been oxidized by periodate. Together, these manipulations lead to a loss of anticoagulant activity along with adequate de-polymerization of the heparin chain.

Further, the resulting reducing end terminal at the site of cleavage is reduced by NaBH₄, which converts the terminal aldehyde to the corresponding diols which are more stable. Subsequently, additives, impurities and side-products are removed by repeated precipitations with ethanol, filtration and centrifugations. Thereafter the substance is obtained in powder form by drying with vacuum and heat. The drug substance will be dissolved in a sterile aqueous buffer to yield the drug product, which is intended for intravenous or subcutaneous administration.

The processes so far described generally include the steps of oxidation, polymer cleavage (alkaline hydrolysis) and reduction. The processes according to the present invention are developed in order to counteract or eliminate any type of non-specific depolymerization of the heparin chains. Non-specific polymerization in this context means generally such depolymerization that is not related to the specific alkaline beta-elimination reaction. Non-specific depolymerization results in structural instabilities of the product that may result in further depolymerization and discoloration during storage of the purified product. In addition, it may contribute to the appearance of atypical species appearing in NMR spectra not normally found in heparin.

The processes described and exemplified in the following section include different aspects of counteracting or eliminating non-specific depolymerization.

### Example 1

Oxidation of Non-Sulfated Glucuronic- and Iduronic Acid (Residues), Deletion of AT-Binding Pentasaccharide and Anticoagulant Activity

A quantity of about 3000 grams of Heparin is dissolved in purified water to obtain a 10-20% w/v solution. The pH of this solution is adjusted to 4.5-5.5. The sodium metaperiodate (NaIO₄) is subsequently added to the process solution; quantity of periodate 15-25% of the weight of heparin. The pH is again adjusted to 4.5-5.5. The reaction is protected from light. The process solution is reacted during the 18-24 hours with constant stirring maintenance of the temperature at 13-17° C., while the temperature is reduced to 5° C. during the last two hours.

Termination of the Oxidation Reaction and Removal of Iodine-Containing Compounds

Ethanol (95-99.5%) is added to the reaction mixture over a period of 0.5-1 hour, with careful stirring and at a temperature of 5-25° C. The volume of ethanol to be added is in the range 1-2 volumes of ethanol per volume of process solution. The oxidized heparin is then allowed to precipitate and sediment for 15-20 hours, after which the mother liquor is decanted and discarded.

Next, the sediment is dissolved in purified water to obtain a 15-30% w/v process solution. NaCl is added to obtain a concentration of 0.15-0.30 mol/liter in the process solution. Stirring continues for another 0.5-1 hour while maintaining the temperature of 5-25° C. Subsequently 1.0-2.0 volumes of ethanol (95-99.5%) per volume of process solution are added to this solution with stirring, during a period of 0.5-1 hour. This precipitates the product from the solution.

De-Polymerization of Polysaccharide Chains by an Alkaline Beta Elimination Process

After the mother liquor has been decanted and discarded, the sediment is stirred in approximately 7 litres of water until completely dissolved, the concentration of the solution is now 15-30%. While maintaining the temperature at 5-25° C. a 4 M NaOH solution is added slowly until a pH of 10.5-12 is obtained. The reaction is initiated and proceeds for 15-95 minutes. At this time, the pH of the solution is recorded and 4 M HCl is added slowly until a pH of 5.5-7 is obtained.

Reduction of Reducing End Terminals

While maintaining the temperature at 13-17° C., the pH of the solution is adjusted to 5.5-6.5. A quantity of 130-150 grams of sodium borohydride is then added to the solution while the pH will increase to 10-11, the reaction is continued for 14-20 hours. After this reaction time, a dilute acid is added slowly in order to adjust the pH to a value of 4, this degrades remaining sodium borohydride. After maintaining a pH of 4 for 45-60 minutes, the pH of the solution is adjusted to 7 with a dilute NaOH solution.

The purification continues according to example 5

### Example 2

Oxidation of Glucuronic and Iduronic Acid (Residues), Deletion of Anticoagulant Activity

A quantity of about 3000 grams of Heparin is dissolved in purified water to obtain a 10-20% w/v solution. The pH of this solution is adjusted to 4.5-5.5. The sodium metaperiodate (NaIO₄) is subsequently added to the process solution; quantity of periodate 15-25% of the weight of heparin. The pH is again adjusted to 4.5-5.5. The reaction is protected from light. The process solution is reacted during the 22-26 hours with constant stirring and maintenance of the temperature at 13-17° C., while the temperature is reduced to 5° C. during the last two hours. The pH at the end of the reaction period is measured and recorded.

Termination of the Oxidation Reaction and Removal of Iodine-Containing Compounds

Ethanol (95-99.5%) is added to the reaction mixture over a period of 0.5-1 hour, with careful stirring and at a temperature of 5-25° C. The volume of ethanol to be added is in the range 1-2 volumes of ethanol per volume of process solution. The oxidized heparin is then allowed to precipitate and sediment for 15-20 hours, after which the mother liquor is decanted and discarded.

De-Polymerization of Polysaccharide Chains by an Alkaline Beta Elimination Process

After the mother liquor has been decanted and discarded, the sediment is stirred in approximately 7 litres of water until it appears visually to be completely dissolved.

While maintaining the temperature at 20-25° C. 4 M NaOH is added slowly until a pH of 10.5-12 is obtained and the reaction thus initiated is allowed to proceed for 15-95 minutes. At this time, the pH of the solution is recorded and 4 M HCl is added slowly until a pH of 5.5-7 is obtained.

Reduction of Reducing End Terminals

After the mother liquor has been decanted and discarded, the sediment is dissolved by addition of purified water until a concentration of the process solution of 15-30% w/v is obtained. While maintaining the temperature at 13-17° C., the pH of the solution is adjusted to 5.5-6.5. A quantity of 130-150 grams of sodium borohydride is then added to the solution and dissolved, the pH will immediately increase to a pH of 10-11, the reaction is continued for 14-20 hours. The pH of the solution, both prior to and after this reaction period, is recorded. After this reaction time, a dilute acid is added slowly in order to adjust the pH to a value of 4, this degrades remaining sodium borohydride. After maintaining a pH of 4 for 45-60 minutes, the pH of the solution is adjusted to 7 with a dilute NaOH solution.

Purification continues according to Example 5.

### Example 3

Oxidation of Glucuronic and Iduronic Acid (Residues), Deletion of Anticoagulant Activity

A quantity of about 3000 grams of Heparin is dissolved in purified water to obtain a 10-20% w/v solution. The pH of this solution is adjusted to 4.5-5.5. The sodium metaperiodate (NaIO₄) is subsequently added to the process solution, quantity of periodate 15-25% of the weight of heparin. The pH is again adjusted to 4.5-5.5. The reactor is protected from light. The process solution is reacted during the 18-24 hours with constant stirring maintenance of the temperature at 13-17° C., while the temperature is reduced to 5° C. during the last two hours.

De-Polymerization of Polysaccharide Chains by an Alkaline Beta Elimination Process

While maintaining the temperature at 5-25° C., 4 M NaOH solution is added slowly until a pH of 10.5-12 is obtained. The reaction is initiated and proceeds for 15-95 minutes. At this time, the pH of the solution is recorded and 4 M HCl is added slowly until a pH of 5.5-7 is obtained.

Reduction of Reducing End Terminals

While maintaining the temperature at 13-17° C., the pH of the solution is adjusted to 5.5-6.5. A quantity of 130-200 grams of sodium borohydride is then added to the solution while the pH will increase to 10-11, the reaction is continued for 14-20 hours. After this reaction time, a dilute acid is added slowly in order to adjust the pH to a value of 4, this degrades remaining sodium borohydride. After maintaining a pH of 4 for 45-60 minutes, the pH of the solution is adjusted to 7 with a dilute NaOH solution.

Precipitation of Reduced Product and Initial Removal of Iodine-Containing Compounds

Ethanol (95-99.5%) is added to the reaction mixture over a period of 0.5-1 hour, with careful stirring and at a temperature of 5-25° C. The volume of ethanol to be added is in the range 1-2 volumes of ethanol per volume of process solution. The oxidized heparin is then allowed to precipitate and sediment for 15-20 hours, after which the mother liquor is decanted and discarded.

Next, the sediment is dissolved in purified water to obtain a 15-30% w/v process solution. NaCl is added to obtain a concentration of 0.15-0.30 mol/liter in the process solution

Purification continues according to Example 5.

### Example 4

Oxidation of Glucuronic and Iduronic Acid (Residues), Deletion of Anticoagulant Activity

A quantity of about 3000 grams of Heparin is dissolved in purified water to obtain a 10-20% w/v solution. The pH of this solution is adjusted to 4.5-5.5. The sodium metaperiodate (NaIO₄) is subsequently added to the process solution, quantity of periodate 15-25% of the weight of heparin. The pH is again adjusted to 4.5-5.5. The reactor is protected from light. The process solution is reacted during the 18-24 hours with constant stirring maintenance of the temperature at 13-17° C., while the temperature is reduced to 5° C. during the last two hours. Next, glycerol is added to quench the reaction, i.e. to convert residual periodate to iodate, 150-200 ml of a 85% glycerol solution is added and reacted for 30-60 minutes while stirring.

Precipitation of Product Removal of Iodine-Containing Compounds and Quencher/Reaction Products

Ethanol (95-99.5%) is added to the reaction mixture over a period of 0.5-1 hour, with careful stirring and at a temperature of 5-25° C. The volume of ethanol to be added is in the range 1-2 volumes of ethanol per volume of process solution. The oxidized heparin is then allowed to precipitate and sediment for 15-20 hours, after which the mother liquor is decanted and discarded.

Next, the sediment is dissolved in purified water to obtain a 15-30% w/v process solution. NaCl is added to obtain a concentration of 0.15-0.30 mol/liter in the process solution. Stirring continues for another 0.5-1 hour while maintaining the temperature of 5-25° C. Subsequently 1.0-2.0 volumes of ethanol (95-99.5%) per volume of process solution are added to this solution with stirring, during a period of 0.5-1 hour. This precipitates the product from the solution.

De-Polymerization of Polysaccharide Chains by an Alkaline Beta Elimination Process

After the mother liquor has been decanted and discarded, the sediment is stirred in approximately 7 litres of water until it appears visually to be completely dissolved. While maintaining the temperature at 5-25° C. 4 M NaOH is added slowly until a pH of 10.5-12 is obtained and the reaction thus initiated is allowed to proceed for 60-95 minutes. At this time, the pH of the solution is recorded and 4 M HCl is added slowly until a pH of 5.5-7 is obtained.

Reduction of Reducing End Terminals

After the mother liquor has been decanted and discarded, the sediment is dissolved by addition of purified water until a concentration of the process solution of 15-30% w/v is obtained. While maintaining the temperature at 13-17° C., the pH of the solution is adjusted to 5.5-6.5. A quantity of 130-150 grams of sodium borohydride is then added to the solution and dissolved, the pH will immediately increase to a pH of 10-11, the reaction is continued for 14-20 hours. The pH of the solution, both prior to and after this reaction period, is recorded. After this reaction time, a dilute acid is added slowly in order to adjust the pH to a value of 4, this degrades remaining sodium borohydride. After maintaining a pH of 4 for 45-60 minutes, the pH of the solution is adjusted to 7 with a dilute NaOH solution.

Purification proceeds according to Example 5.

### Example 5

Purification of the Product

Removal of Process Additives and Impurities, Addition of Counter-Ions and Filtration

Process solutions according to Examples 1-4 arriving from the final chemical modification step of reducing the end terminals by borohydride is worked up according the methodologies outlined below.

One volume of process solution is then added to 1.5-2.5 volumes of ethanol (95-99.5%) followed by centrifugation at >2000 G, at <20° C. for 20-30 minutes, after which the supernatant is decanted and discarded.

The product paste obtained by centrifugation is then dissolved in purified water to obtain a product concentration 10-20% w/v. Then NaCl is added to obtain a concentration of 0.20-0.35 mol/liter. Next 1.5-2.5 volumes of ethanol (95-99.5%) are added per volume of process solution which precipitates the product from the solution. Centrifugation follows as described above

Next the remaining paste is added purified water to dissolve. The product concentration would now be in the range of 10-20% w/v. The pH of the product solution is now adjusted to 6.5-7.5. The solution is then filtered to remove any particulates. Then, to one volume of process solution is added 1.5-2.5 volumes of ethanol (95-99.5%). Centrifugation follows at >2000 G, and at <20° C. for 20-30 minutes after which the supernatant is decanted and discarded.

Dewatering of Precipitate Paste and Reduction of Particle Size.

A reactor is filled with ethanol, volume about 2 liters. While stirring the ethanol, the precipitate paste is added. The mechanical stirring solidifies the paste and replaces the water present by the ethanol giving a homogenous particle suspension. The stirring is discontinued after 1-2 hours after which the particles are allowed to sediment. After removal of excessive liquid, the particles are passed through a sieve or a mill to obtain smaller and uniform sized particles.

Drying of Product

The product is distributed evenly onto trays, and placed in a vacuum cabinet. Vacuum is applied and heating is performed at 35-40° C. A stream of nitrogen is passed through the drier at this time while maintaining the low pressure in the dryer. When a constant weight is obtained of the product, i.e. no further evaporation is noticed, the drying is considered complete. The product is packed and protected from humidity.

### Example 6

Oxidation of Glucuronic and Iduronic Acid (Residues), Deletion of Anticoagulant Activity

A quantity of about 3000 grams of Heparin is dissolved in purified water to obtain a 10-20% w/v solution. The pH of this solution is adjusted to 4.5-5.5. The sodium metaperiodate (NaIO₄) is subsequently added to the process solution, quantity of periodate 15-25% of the weight of heparin. The pH is again adjusted to 4.5-5.5. The reaction is protected from light. The process solution is reacted during the 18-24 hours with constant stirring maintenance of the temperature at 13-17° C., while the temperature is reduced to 5° C. during the last two hours.

De-Polymerization of Polysaccharide Chains by an Alkaline Beta Elimination Process

While maintaining the temperature at 5-25° C. 4 M NaOH is added slowly until a pH of 10.5-12 is obtained and the reaction thus initiated is allowed to proceed for 15-95 minutes. At this time, the pH of the solution is recorded and 4 M HCl is added slowly until a pH of 5.5-7 is obtained.

Reduction of Reducing End Terminals

After the mother liquor has been decanted and discarded, the sediment is dissolved by addition of purified water until a concentration of the process solution of 15-30% w/v is obtained. While maintaining the temperature at 13-17° C., the pH of the solution is adjusted to 5.5-6.5. A quantity of 130-200 grams of sodium borohydride is then added to the solution and dissolved, the pH will immediately increase to a pH of 10-11, the reaction is continued for 14-20 hours. The pH of the solution, both prior to and after this reaction period, is recorded. After this reaction time, a dilute acid is added slowly in order to adjust the pH to a value of 4, this degrades remaining sodium borohydride. After maintaining a pH of 4 for 45-60 minutes, the pH of the solution is adjusted to 7 with a dilute NaOH solution. Purified water is now added to the solution until a conductivity of 15-20 mS/cm is obtained of the reaction solution.

Purification of Product by Anion Exchange Chromatography

A column with a diameter 500 mm is packed with media, DEAE-Sepharose or QAE-Sepharose to a volume of 25-30 liters corresponding to a bed height of 10-15 cm. The chromatography is performed in 3-4 cycles to purify all the product.

Next buffers are prepared,

Equilibration buffer, Buffer A, 15 mM phosphate, 150 mM NaCl

Elution buffer, Buffer B, 2 M NaCl solution

Sanitation buffer, 0.5 M NaOH

The chromatography step is performed at 15-25° C., at flow rate of ≦200 cm/hour or approx. 350 liters/hour.

The column is equilibrated with the equilibration buffer until the eluent has a conductivity of 15-20 mS/cm. Next the oxidized heparin solution is pumped into the column. The quantity of crude product to be applied corresponds to <40 g/liter of chromatography media.

An isocratic wash follows with equilibration buffer and is discontinued when the UV 210-254 nm has reached a baseline. Typically 5 bed volumes of buffer are required to reach baseline. Chemicals added to the process and products formed of these are removed.

Next, the ionic strength of the buffer applied onto the column is linearly increased by performing a gradient elution. The Buffer A decreases from 100% to 0% replaced by 100% Buffer B over 5 bed volumes. The product, eluate is collected when the UV absorbance is >0.1 AU and is discontinued when the signal is <0.1 AU. Sanitation of the column is then performed after which it is again prepared for the next cycle of chromatography. Eluates from all runs are combined and stored at 15-25° C.

De-Salting of the Product

One volume of the combined eluates from previous step is added 3 volumes of 95-99.5% ethanol, 15-25° C., under constant stirring. This precipitates the product out of solution. The product is allowed to sediment for >3 hours. Next, the sediment is dissolved in purified water to a concentration of 15-25%. The solution is now added to cold ethanol (<−5° C.) 95-99.5%, typically 5 volumes of ethanol per one volume of product solution are consumed. Next follows centrifugation in a continuous mode, >2000 G, the product paste is thereafter collected and prepared for drying.

Drying of Product

The product is distributed evenly onto trays, and placed in a vacuum cabinet. Vacuum is applied and heating is performed at 35-40° C.A stream of nitrogen is passed through the drier at this time while maintaining the low pressure in the dryer. When a constant weight is obtained of the product, i.e. no further evaporation is noticed, the drying is considered complete. The product is milled and made homogenous, thereafter packed and protected from humidity.

### Example 8

Low anticoagulant heparin produced according to the examples 1 and 3 was subjected to 1H-NMR analysis and compared to the spectrum of native heparin.

Table II demonstrates signals in the interval 5.00 ppm to 6.50 ppm not present in native heparin generated from non-reducing end unsaturated glucosamines. The results of Table II show that it is possible to reduce the presence of such compounds not predicted to be present in spectrum from native heparin to low levels. In comparison, the current limit applicable to heparin quality control, monograph 7, EDQM is <4% compared to the signal at 5.42 ppm for any signal in the region 5.70-8.00 ppm.

TABLE II
Qualitative results of a low anticoagulant heparin with regards to unusual
signals. Signal intensity for signals 6.15 and 5.95 ppm in a 1H-NMR
spectra

Intensity (% ratio) to 5.42 ppm
signal of a native heparin
following EDQM, monograph 7

| | Production | 6.15 ppm | 5.95 ppm |
|---|---|---|---|
| Sample | method | % of ref. signal | % of ref. signal |
| Batch 1 | Example 1 | 11 | 12 |
| Batch 2 | Example 1 | 13 | 16 |
| Batch 3 | Example 3 | 2 | 2 |

Further, the presence of non reducing end unsaturated glucosamines was also quantified by combined 1H-NMR and 13C-NMR spectra evaluation(HSQC) and demonstrated as mol % of total glucosamines (see Table III).

Furthermore, the sample was analyzed by following the NMR two-dimensional (2D) method involving the combined use of proton and carbon NMR spectroscopy (HSQC) as previously described (see Guerrini M., Naggi A., Guglieri S, Santarsiero R, Torri G. Anal Biochem 2005; 337, 35-47.)

Table III demonstrates the fraction (%) of modified glucosamines compared to the total amount of glucosamines of the low anticoagulant heparin as present as signals at 5.95 ppm and 6.15 ppm in the ¹H-NMR spectrum.

TABLE III
Results from quantitative determination of unusual signals 5.95 ppm,
6.15 ppm of total glucosamine

| | | 6.15 ppm signal | 5.95 ppm signal |
|---|---|---|---|
| | Production | mol % of | mol % of |
| Sample | method | glucosamine | glucosamine |
| Batch 1 | Example 1 | 6 | 3 |
| Batch 2 | Example 3 | <1 | <1 |

### Example 9

The product manufactured according to any one of the examples above can prepared as drug product by a conventional aseptic process, such as solution comprising 150 mg/mL of active product and Na phosphate to 15 mM, pH 6-8. The so obtained drug product is intended primarily for subcutaneous administration but suitable for intra-venous administration.

The resulting product is a depolymerized form of heparin with a projected average molecular weight of 4.6-6.9 kDa and with essentially no anticoagulant activity.

The product has a size distribution of polysaccharide polymers, with a range for n of 2-20 corresponding to molecular weights of 1.2-15 kDa. The predominant size is 6-16 disaccharide units corresponding to molecular weights of 3.6-9.6 kDa.

The molecular weight was determined by GPC-HPLC carried out with a TSK 2000 and TSK 3000 SW columns in series. Refractive index was used for evaluation. First international calibrant for LMWH was used.

Below is presented the molecular mass distribution and the corresponding part of the cumulative percentage of total weight.

TABLE IV
Distribution of polysaccharides and their corresponding molecular
mass in as cumulative % of weight for several batches

| Molecular mass, | |
|---|---|
| kDa | Cumulative weight, % |
| >15 | <1 |
| >10 | 4-15 |
| >9 | 7-20 |
| >8 | 10-27 |
| >7 | 15-35 |
| >6 | 22-45 |
| >5 | 34-56 |
| >4 | 47-70 |
| >3 | >70 |
| >2 | >85 |

The corresponding value for weight average molecular weight, Mw falls in the range 4.6-6.9 kDa

### Example 10

The stability of the drug substance (powder) and drug product dissolved in aqueous phosphate buffered solution of a chemically modified heparin produced in accordance with Examples 1 to 3 and formulated in accordance with Example 9 was studied for stability over 36 months at ambient temperature. The initial product was clear white to slight yellow solution had an absorbance at 400 nm (10% w/v solution) of 0.14, a pH of 7.0 and osmolality of 658 mOsm/kg, an average molecular weight of 5.6 kDa and a content of 150 mg/ml.

After 36 months, the drug product had the same visual appearance, an absorbance at 400 nm (10% w/v solution) of 0.13, a pH of 7.1 and osmolality of 657 mOsm/kg, an average molecular weight of 5.4 kDa and a content of 153 mg/ml.

(Example 10 is rewritten to depend on one summarized stability test)

### Example 11

Subcutaneous Administration

Chemically modified heparin produced by the method disclosed in Example 1 and labeled with tritium was administered to Sprauge Dawley rats and dogs.

Results:

Following subcutaneous administration at 2, 8 and 24 mg chemically modified heparin /kg/day in the rat and 3, 15 and 45 mg chemically modified heparin/kg/day in the dog, absorption was rapid and maximal plasma levels were generally reached within 0.5 and 1.5 h in the rat and dog, respectively. The subcutaneous bioavailability was around 90% in both the rat and the dog. The corresponding bioavailability for heparin is about 10%.

### Example 12

Treatment with DF01 During Late Pregnancy

Study Design

This was a randomised, double blind, placebo-controlled, multicentre study to assess the safety and efficacy of pre-treatment with DF01 during late pregnancy in reducing labor time. Eighteen study centres in Sweden participated in the study.

DF01 is a chemically modified heparin according to the invention that is low-anticoagulant heparin chemically generated by periodate oxidation of heparin from pig intestinal mucosa, followed by β-elimination of the product, following Examples 1 and 9.

The protocol stated that each subject would come to the clinic daily from the treatment start at a gestational age from week 38+0 up to week 40+0 until labor to receive a s.c. injection of the investigational medicinal product. The anticipated duration of participation in the study was 1-28 days (+screening and follow-up periods) for each subject. All women had to be induced into labor at the latest at 42+0 weeks of gestation. A maximum of 28 days of treatment [maximally 28 doses of the investigational medicinal product (IMP)] was given. A follow-up visit was to take place at 8-16 weeks after delivery.

Treatments

DF01 is a depolymerized heparin that is essentially deprived of its anticoagulant activity (<10 IU/mg by pharmacopoeial anti-factor Xa- and anti-factor IIa assays). The weight average Mw is 5 000-7 000.

DF01 and matching placebo, were provided as solutions for subcutaneous injection.

The pharmaceutical preparation of DF01 is a solution for subcutaneous injection, 8 mL dispensed in glass vials sealed with a rubber stopper and covered with a tear-off aluminum cap.

Each mL of the DF01 solution contains the following:
DF01, 150 mg
Phosphate buffer, 0.015 M
Benzyl alcohol, 14 mg.

A sterile physiological sodium chloride solution preserved with benzyl alcohol was used as placebo. Eight (8) mL of the placebo were provided in vials in the same way as for the drug product.

Each mL of the placebo solution contains the following:
Sodium chloride, 9 mg
Benzyl alcohol, 14 mg.

The subjects received 60 mg/day of DF01 (0.4 mL) (corresponding to 1.00 mg/kg/day in a 60 kg subject) or placebo (0.4 mL).

The products was administered by daily subcutaneous injections with treatment start at gestational age of week 38+0 to week 40+0 and treatment duration until labor. If still undelivered at 42+0 labor was to be induced. The maximum duration of treatment was 28 days. The allowed time interval between the daily injections was 24+/−6 hours, i.e. 18-30 hours. If the time limits were occasionally not met or a dose missed, the treatment could still continue.

Results

1. Vaginal deliveries with induction of labor

There were a total of 30 non-Caesarean deliveries with induction of labor in different ways and a defined start of labor (14 in the DF01 group and 16 in the Placebo group), see FIG. 1; Product-limit Birth Curve, vaginal deliveries with induction of labor.

The log-rank test showed a significant difference between the treatment groups with a p-value of 0.0041. The birth rate ratio assessed from the Cox proportional hazard model was 3.365 (95% Cl 1.428-8.341). As shown in FIG. 1, women induced into labor and pre-treated with DF01 had a significant shorter delivery time compared to women induced into labor but who did not receive DF01 treatment prior to labor. None of the women on DF01 had a protracted labor and all neonates were healthy.

2. Vaginal deliveries with induction of labor where the women received prostaglandin E2

Of the 30 non-Caesarean deliveries shown FIG. 1, a total of 12 non-caesarean deliveries labor was induced with prostaglandin E2 (7 in the DF01 group and 5 in the Placebo group), see FIG. 2; Product-limit Birth Curve. The log-rank test showed a significant difference between the treatment groups with a p-value of 0.033 (Median: DF01: 5.7; Median: Placebo 8.9). The results indicate that DF01 supports the capacity of prostaglandins to promote cervical ripening and that women who both received DF01 and prostaglandin E2 have a significantly shorter delivery time than women who only received prostaglandin E2.

3. Vaginal deliveries with induction of labor where the women received oxytocin

Of the 30 non-Caesarean deliveries with induction of labor, the women received oxytocin (7 in the DF01 group and 11 in the Placebo group), see FIG. 3 Product-limit Birth Curve. The log-rank test showed a significant difference between the treatment groups with a p-value of 0.0336 (Median: DF01: 3.7; Median: Placebo 6.4). This indicates a lower need of oxytocin with DF01 and thus an advantage due to the well-known side effects (e.g. myometrial hypercontractility) of high dose administration of oxytocin.

A treatment regimen in the case of labor induction will therefore typically entail a directly intervening treatment with DF01 followed by methods triggering the release (balloons/rupturing of membranes) of endogenous oxytocin or the administration of exogenous oxytocin.

### Example 13

Human uterine smooth muscle cells were established in a culture. A method to measuring intracellular Ca²⁺ with the calcium indicator dye Fluo-4 and live cell imaging with confocal microscopy was established for the cells. The cells were treated with oxytocin and a Ca²⁺-influx to the cytosol was demonstrated.

The effect was dose-dependent with a maximum effect already at 0.05 IU/ml oxytocin. For the experiments, DF01 as described in Example 12 was used.

FIG. 4A shows that DF01 alone did not affect the Ca²⁺-concentration. However, when DF01 was given together with oxytocin, an increased and sustained Ca²⁺-level was attained compared to oxytocin alone, see FIG. 4B and FIG. 4C. The dose response pathway, see FIG. 4D, shows that the effect of DF01 correlates with the amount of Ca²⁺-peaks. The results demonstrate a mechanism for how DF01 exert an effect on uterine contraction by promoting and sustaining the effect of oxytocin.

The mechanism was further investigated by preincubating uterine smooth muscle cells with 10 μM of verapamil for 30 min. Verapamil did not affect the Ca²⁺ influx, induced by either oxytocin or by the combination of oxytocin and DF01. It can therefore be concluded s that L-channels not are involved.

It was further investigated if the main transport mechanism of inositol-3 phosphate (IP3) stimulated Ca²⁺ transport of the endoplasmatic reticulum. To study this pathway, 2-Aminoethoxydiphenyl borate (2-APB) was tested on Ca²⁺ after 30 min of incubation with a concentration of 100 μM. This inhibitor decreased strongly both the oxytocin and the oxytocin/DF01 stimulated Ca²⁺-transport.

To further characterize the interaction between oxytocin and DF01 the effect of the oxytocin receptor inhibitor Atosiban was used and the cells subjected to the DF01 enhanced oxytocin effect on Ca²⁺ transport. Atosiban in a concentration of 10⁻⁶ M clearly inhibited the effect of both oxytocin and the combination oxytocin/DF01

The results indicate that DF01 does not by itself effect Ca²⁺-transport. However in combination with oxytocin a clear dose response enhanced stimulation of Ca²⁺ transport is noted. DF01 stabilizes the effect of oxytocin resulting in longer periods of stimulation. The effect of does not involve L-channels but rather involves IP3 stimulated Ca²⁺ influx in oxytocin signaling. The effect of the oxytocin antagonist suggests that the effect on DF01 operates on the oxytocin receptor level.

It is concluded that DF01 and chemically modified heparin or heparan sulfates according to the invention are useful agents to administer for directly improving myometrial contractions of the uterus and to directly and interveningly treat complications associated with inadequate or absent myometrial contractions. In summary, DF01 and similar chemically modified heparin or heparan sulfate and heparin sulfates are concluded to be effective directly in intervening treatments required to induce labor

Although particular embodiments have been disclosed herein in detail, this has been done by way of example for purposes of illustration only, and is not intended to be limiting with respect to the scope of the appended claims that follow. In particular, it is contemplated by the inventors that various substitutions, alterations, and modifications may be made to the invention without departing from the spirit and scope of the invention as defined by the claims.

1. A kit comprising:
a chemically modified heparin or heparan sulfate in combination with an agent capable of promoting cervical ripening or promoting myometrial contractions of the uterus, the chemically modified heparin or heparan sulfate having an antifactor IIa activity of less than 10 IU/mg, an antifactor Xa activity of less than 10 IU/mg, and an average molecular weight (Mw) from about 4.6 to about 6.9 kDa, and comprising:
(i) polysaccharide chains essentially free of chemically intact saccharide sequences mediating the anticoagulant effect; and
(ii) polysaccharide chains corresponding to molecular weights between 1.2 and 12 kDa with a predominantly occurring disaccharide according to (Formula I), 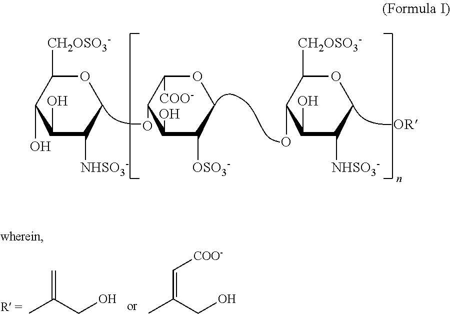
n is an integer from 2 to 20
wherein the chemically modified heparin or heparan sulfate comprises non-reducing end unsaturated glucosamines that are presented as signals in the interval of 5.0 to 6.5 ppm in a ¹H-NMR spectrum with an intensity (% ratio) of less than 4% in relation to the signal at 5.42 ppm from native heparin.

2. The kit according to claim 1 wherein the agent for promoting cervical ripening is present.

3. The kit according to claim 2, wherein the agent for promoting cervical ripening comprises of a prostaglandin.

4. The kit according to claim 3, wherein the prostaglandin is selected from the group consisting of dinoproston (PGE2) and misoprostol.

5-8. (canceled)

9. The kit according to claim 1, wherein the predominantly occurring polysaccharide chains have between 6 and 12 disaccharide units with molecular weights from 3.6 to 7.2 kDa.

10. The kit according to claim 1, wherein at least 70% of the polysaccharide chains have a molecular weight above 3 kDa.

11. The kit according to claim 1, wherein the chemically modified heparin or heparan sulfate has a distribution of polysaccharides and their corresponding molecular mass expressed as cumulative % of weight according to the table:

| Molecular mass, | |
|---|---|
| kDa | Cumulative weight, % |
| >10 | 4-15 |
| >8 | 10-25 |
| >6 | 22-45 |
| >3 | >70 |

12-13. (canceled)

14. The kit according to claim 1, wherein the modified glucosamine signals are present at 5.95 ppm and 6.15 ppm in the ¹-NMR spectrum.

15. The kit according to claim 1, wherein the non-reducing end unsaturated glucosamines comprise less than 1% of the total content of glucosamines.

16. (canceled)

17. The kit according to claim 1, wherein the chemically modified heparin or heparan sulfate is essentially free of intact non-sulfated iduronic and/or glucuronic acids.

18. A method for the induction of labor in women, comprising:
administering to a pregnant woman an effective amount of a chemically modified heparin or heparan sulfate with an antifactor IIa activity of less than 10 IU/mg, an antifactor Xa activity of less than 10 IU/mg, and an average molecular weight (Mw) from about 4.6 to about 6.9 kDa, and comprising:
(i) polysaccharide chains essentially free of chemically intact saccharide sequences mediating the anticoagulant effect; and
(ii) polysaccharide chains corresponding to molecular weights between 1.2 and 12 kDa with a predominantly occurring disaccharide according to (Formula I), 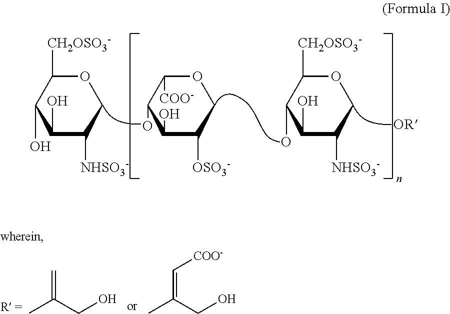
n is an integer from 2 to 20,
wherein the chemically modified heparin or heparan sulfate comprises non-reducing end unsaturated glucosamines that are presented as signals in the interval of 5.0 to 6.5 ppm in a ¹H-NMR spectrum with an intensity (% ratio) of less than 4% in relation to the signal at 5.42 ppm from native heparin; and
administering to the pregnant woman an agent capable of promoting cervical ripening or promoting myometrial contractions of the uterus.

19-20. (canceled)

21. The method according to claim 18, wherein said administering the chemically modified heparin or heparan sulfate is subsequent to said administering the agent capable of promoting cervical ripening or promoting myometrial contractions of the uterus.

22. The method according to claim 18, wherein the pregnant woman has an unripe cervix and the agent capable of promoting cervical ripening is administered.

23. The method according to claim 22, wherein the agent capable of promoting cervical ripening is a prostaglandin.

24. The method according to claim 23, wherein the prostaglandin is selected from the group consisting of dinoproston (PGE2) and misoprostol.

25. The kit according to claim 1, wherein the kit comprises both the agent capable of promoting cervical ripening and the agent promoting myometrial contractions of the uterus.
